(19) United States
(12) Patent Application Publication
(10) Pub. No.: US 2023/0257806 A1
(43) Pub. Date: Aug. 17, 2023

(54) SYSTEM AND METHOD FOR ON-SITE AUTHENTICATION OF NUCLEIC ACID TAGGANTS USING NUCLEIC ACID LATERAL FLOW DEVICE

(71) Applicant: Applied DNA Sciences, Inc., Stony Brook, NY (US)

(72) Inventor: Lawrence Jung, Dix Hills, NY (US)

(73) Assignee: Applied DNA Sciences, Inc., Stony Brook, NY (US)

(21) Appl. No.: 18/110,659

(22) Filed: Feb. 16, 2023

Related U.S. Application Data
(60) Provisional application No. 63/310,732, filed on Feb. 16, 2022.

(51) Int. Cl. C12Q 1/686

(52) U.S. Cl. CPC C12Q 1/686

(57) ABSTRACT

A system and method for interrogating objects containing a nucleic acid taggant including a sample collection device for obtaining a sample containing a nucleic acid taggant from an object. The at least one taggent includes at least one predetermined nucleic acid sequence. A reagent contacts the sample. A polymerase chain reaction (PCR) device for device for receiving the sample and amplifying the at least one nucleic acid sequence contained in the sample to produce a PCR sample solution. A Nucleic Acid Lateral Flow (NALF) device has a sample receiving end for receiving the PCR sample solution and having an indication end. The indication end includes a first test area and at least one control area. The first test area provides a visual indication upon the detection of a first predetermined nucleic acid sequence carried in the PCR sample solution.

This application claims the benefit of priority to U.S. Provisional Patent Application No. 63/310,732 filed on Feb. 16, 2022, the entire content of which is incorporated by reference herein.

### FIELD OF TECHNOLOGY

The present disclosure is directed to a system and method for on-site authentication of nucleic acid taggants using nucleic acid lateral flow device and in particular for on-site interrogation of products marked with nucleic acid taggants using the polymerase chain reaction (PCR) and a nucleic acid lateral flow (NALF) device.

### BACKGROUND

Manufacturers have an interest in protecting the authenticity, integrity and purity of their products that are fabricated from quality components and may be subject to mixing or dilution with less expensive, lower quality materials. Such adulteration and even outright counterfeit substitution of process feed-stocks and production materials, received from suppliers to be processed by the manufacturers, often escapes detection until after the products are manufactured. In addition, certain goods, such as cannabis based products, are subject to regulatory control and their provenance must be verified along the stream of commerce.

Tags have been applied to goods, which contain information that can be used for verification and authentication. Tags have included taggants containing nucleic acid material. Despite being composed of relatively simple nucleotide building blocks, nucleic acids are capable of encoding a vast array of information: witness the human genome encodes all the information necessary for the synthesis and assembly of all the components of the human body from the neural networks of the brain to the intricate structures of the skeleton, tissues and organs. Nucleic acids include deoxyribonucleic acid (DNA) and the more labile ribonucleic (RNA). Since nucleic acid sequences can be unique and complex, utilization of these particular characteristics in solving several common coding problems, such as authenticating and tracking products and detecting counterfeit products, has recently attracted great interest.

Nucleic acids, such as, for example, deoxyribonucleic acid (DNA) and ribonucleic acid (RNA) which encode essential hereditary information have been looked to as an improved alternative to commonly used anti-counterfeiting labels and markers. DNA and RNA are polymers consisting of a chain of nucleotides, referred to as “oligonucleotides” consisting of relatively short chains of up to say, twenty to fifty bases in length, or “polynucleotides” for longer chains. These oligonucleotide or polynucleotide chains consist of a number of nucleotides linked together in sequence like beads on a string. Each nucleotide consists of a ribose sugar-phosphate linked to one of only four kinds of nitrogenous bases: adenine (often represented in abbreviated form as “A”), guanine (represented as “G”), cytosine (represented as “C”) and thymine (represented as “T”) in the case of DNA; and adenine (A), guanine (G), cytosine (C) and uracil (U) in the case of RNA. The oligonucleotides or polynucleotides share the same sugar-phosphate backbone. The 3′-hydroxyl group on the ribose sugar is covalently bonded to the 5′-phosphate group of its neighboring nucleotide to form a chain structure with the planar nitrogenous bases protruding from the chain not unlike the teeth of a comb.

The bases A, T, G and C in one oligonucleotides or polynucleotides chain are each capable of specific-pairing with another base a different chain to form a double stranded structure, or with the same chain to form a double stranded loop or hairpin structure: Adenine specifically bonds with thymine through two hydrogen bonds in DNA (or with uracil in RNA) and cytosine specifically bonds with guanine through three hydrogen bonds. That is, T will bond to A and G to C bringing two nucleotide chains together to form a double strand, or two parts of a single nucleotide chain together to form a double stranded region with each strand of the duplex connected by a loop.

An additional advantage of nucleic acids for use as markers or taggants is that with the appropriate proper protection these molecules can be preserved for long periods of time. Evidence from preserved specimens in glaciers, ice sheets, tar pits and bogs and marshes shows that DNA is resilient to degradation over thousands, and in some cases millions of years. Such evidence has been used to deduce information concerning the ancestry and origins of ancient peoples as well as of plants and animals. Protected marker DNA can also be stabilized in polymers for coating of high value articles or objects of interest so as to survive long periods of time and can then used for identification, authentication and tracking purposes. This ability to persist over long periods of time coupled with very sensitive methods to detect low numbers of molecules for instance by amplification using the polymerase chain reaction (PCR), makes nucleic acids, and DNA in particular, an attractive candidate for use as a marker. Moreover, nucleic acids offer an almost unlimited coding capacity since the number of possible unique sequences increases fourfold with every additional base of the sequence of the oligonucleotide or polynucleotide.

Once the PCR process is completed, the amplified segments can be analyzed by gel electrophoresis or sequence specific detection techniques. The resulting amplified segments generated can then be compared to other nucleotide segments from a known source. The PCR-generated nucleotide sequences are then placed next to known nucleotide sequences from humans, pathogens, or other sources in a separating gel. Electrical current is then run through the gel, and the various nucleotide sequences within the gel form bands that resemble a ladder, according to their electrical charge and molecular size. Bands or ladder-like steps that migrate to the same levels in the gel show identity of nucleotide sequences. This method is one conventional way PCR tests are completed.

It is desirable to have samples processed in the field at the site of sample collection. However, nucleic acid processing and identification can be challenging in the field due to the amplification and subsequent analysis, such as use of gel electrophoresis.

Accordingly, it would be desirable to have the ability to collect samples and subject them to ta PCR amplification and identification in the field in order to determine if a particular nucleic acid taggant is present.

### SUMMARY

The present disclosure provides a system for interrogating objects containing at least one nucleic acid taggant including a sample collection device for obtaining a sample containing a nucleic acid taggant from an object. The at least one taggent including at least one predetermined nucleic acid sequence. A reagent contacts the sample. A polymerase chain reaction (PCR) device for device for receiving the sample and amplifying the at least one nucleic acid sequence contained in the sample to produce a PCR sample solution. A Nucleic Acid Lateral Flow (NALF) device has a sample receiving end for receiving the PCR sample solution and having an indication end. The indication end includes a first test area and at least one control area. The first test area provides a visual indication upon the detection of the first predetermined nucleic acid sequence carried in the PCR sample solution.

The present disclosure also provides a method of interrogating an object marked with a nucleic acid taggant including:

obtaining a sample from an object containing at least one nucleic acid taggant, the at least one taggent including at least one predetermined nucleic acid sequence;

placing the sample in a reagent;

amplifying the nucleic acid sequence contained in the sample by a polymerase chain reaction (PCR) device;

obtaining from the PCR device an amplified PCR sample solution including amplified nucleic acid; and

depositing the amplified PCR sample solution on a receiving end of a Nucleic Acid Lateral Flow (NALF) device, the NALF device having an indication end, the indication end including a first test area and at least one control area formed to detect the first predetermined nucleic acid sequence, the first test area providing a visual indication upon the detection of the first predetermined nucleic acid sequence.

The present disclosure further provides a method of tracking the origin of cannabis and cannabis derivative products comprising:

applying at least one nucleic acid taggant to a cannabis plant to create a nucleic acid tagged cannabis plant, the at least one taggant imparting at least one piece of information about the origin of the cannabis plant;

processing the nucleic acid tagged cannabis plant to create nucleic acid tagged cannabis products and/or nucleic acid tagged cannabis derivative products;

interrogating a nucleic acid tagged cannabis product and/or cannabis derivative product to ascertain information from the nucleic acid tag; the interrogation including:
amplifying the nucleic acid contained on the sample by a polymerase chain reaction (PCR) device to produce an amplified PCR sample including a first predetermined nucleic acid sequence; and
depositing the amplified PCR sample on a receiving end of a Nucleic Acid Lateral Flow (NALF) device, to obtain a visual indication upon the detection of a first predetermined nucleic acid sequence.

The disclosure still further provides a system for interrogating objects containing a nucleic acid taggant including a sample collection device for obtaining a sample containing a nucleic acid taggant from an object, and a reagent for contacting the sample. The system includes a polymerase chain reaction (PCR) device for receiving the sample and amplifying the nucleic acid contained on the sample to produce a PCR sample solution. The system further includes a Nucleic Acid Lateral Flow (NALF) device having a sample receiving portion for receiving the PCR sample solution and one or more test areas. Each test area provides a visual indication upon the detection of a predetermined nucleic acid sequence carried in the PCR sample solution. A computing device is in communication with an image scanning device for scanning the NALF device and the visual indications thereon. The computing device is in communication with a database of images. The computing device compares the scanned image with the databases images to find a match. The computing device outputs information about the object from which the sample is obtained responsive to the match.

The disclosure also provides a method for interrogating objects containing a nucleic acid taggant comprising:
collecting with a sample collection device a sample containing a nucleic acid taggant from an object, at least one taggent including at least one predetermined nucleic acid sequence;
contacting the sample with a reagent;
subjecting the sample to a polymerase chain reaction (PCR) process for amplifying the at least one predetermined nucleic acid sequence contained in the sample to obtain an amplified PCR sample;
applying the amplified PCR sample to a Nucleic Acid Lateral Flow (NALF) device having a sample receiving portion and one or more test areas, each test area providing a visual indication upon the detection of a predetermined nucleic acid sequence;
scanning the NALF device and the visual indications thereon with an image scanning device, the scanning device being in operative communication with a computing device;
the computing device being in communication with a database of images, the computing device comparing the scanned image with the databases images to find a match; and

outputting information with the computing device about the object responsive to the match.

### BRIEF DESCRIPTION OF THE DRAWINGS

FIG. 1 is a schematic flow diagram of system using PCR and NALF test device to determine the presence of a nucleic acid taggant.

FIG. 2 is a schematic diagram of a NALF test device.

FIG. 3A is a NALF test device showing results for a cannabis extract.

FIG. 3B is a container of material tested by the device of FIG. 3A.

FIG. 4 is a NALF test device showing multiple test line areas.

FIG. 5A is a NALF test device showing results for a consumer products.

FIG. 5B is a NALF test device showing results for a consumer products.

FIG. 5C is a product including material tested by the device of FIGS. 5A and 5B.

FIG. 6A is a NALF test device showing results for cosmetics and cosmetic ingredients.

FIG. 6B is a NALF test device showing results for cosmetics and cosmetic ingredients.

FIG. 6C is a cosmetic ingredient tested by the device of FIG. 6B.

FIG. 6D is a NALF test device showing results for cosmetics and cosmetic ingredients.

FIG. 6E is a cosmetic product tested by the device of FIG. 6D.

FIG. 7A is a NALF test device showing results for a nutraceuticals and nutraceutical ingredients

FIG. 7B is a nutraceutical product tested by the device of FIG. 7A.

FIG. 8A is a NALF test device showing results for pharmaceuticals and Pharmaceutical Excipient.

FIG. 8B is a pharmaceutical product tested by the device of FIG. 8A.

FIG. 9A is a NALF test device showing results for a product label.

FIG. 9B is a product label tested by the device of FIG. 9B.

FIG. 10 is a NALF array including a plurality of test areas.

FIG. 11 is a schematic of a NALF array being scanned by a mobile device.

FIG. 12 is a illustration of a display presenting sample information derived from a scan of a NALF array of FIG. 11.

FIG. 13 is a flow chart of a method in accordance with the present disclosure.

### DETAILED DESCRIPTION

Utilization of a portable PCR device and a lateral flow device in sequence allows for ease of use for verification and authentication of articles such as goods, consumables, and the like, on-site location where the objects are located and the verification and/or authentication check is desired.

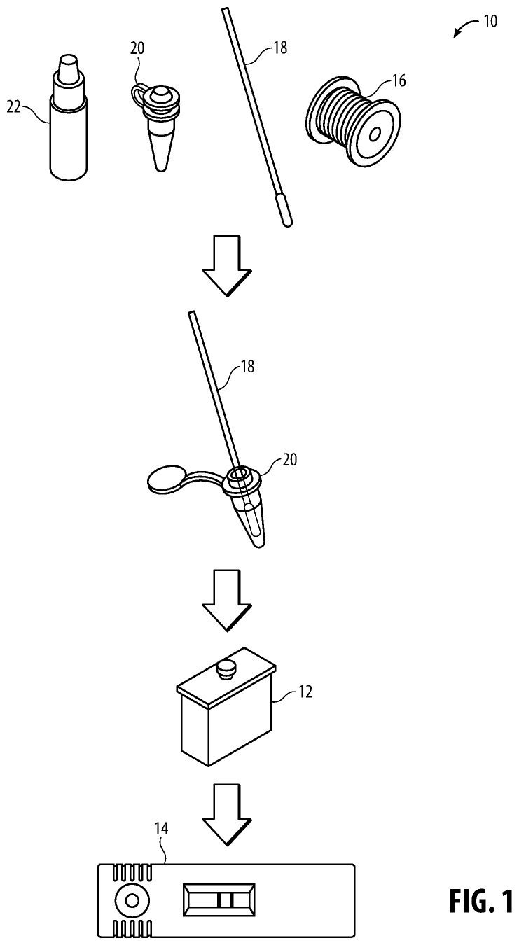

With reference to FIG. 1, the present disclosure provides a system 10 and method to interrogate samples taken from an object or article 16 to authenticate or verify a particular aspect of the article, e.g., quality, origin, or legality. The system 10 includes a sample collection device 18, a portable nucleic acid PCR device 12, and a nucleic acid lateral flow device 14. The system facilitates ease of use in the field and at supply chain nodes such as U.S. Customs & Border Control, or other state and federal agencies.

Articles or objects 16 to be inspected may include clothing, food, agricultural products, such as cannabis or cannabis-derived products, cotton and the like, industrial products, etc. The article 16 would be marked with nucleic acid taggant in a manner known in the art. Such taggant marking is set forth in commonly assigned U.S. Pat. Nos. 8,415,164, 8,426,216, and 9,266,370, the entire contents of each are incorporated by reference herein. While use of the system 10 in association with cannabis is described herein, this is for purposes of example and not intended to be limiting. It is contemplated that this system 10 may be used in association with a number of articles, plant-based or otherwise.

Cannabis is a highly regulated industry with the plant and its byproducts subject to significant restrictions and regulations. For example, it is often necessary to track and trace the origin of the cannabis plant and its byproducts from seed-to plant-to-finished products. With such tracing, the provenance of the finished products can be verified and the regulatory compliance and legality of the goods ascertained. In order to permit such tracking and tracing, it is desirable to be able to so in the field or on-site, that is, at the location where the articles are being inspected or processed. This system avoids the need to sends the articles, or samples thereof, to a separate location for verification. The present system 10 facilitates such on-site interrogation and verification of cannabis.

The articles to be interrogated have a nucleic acid, e.g., RNA or DNA, a taggant, or a plurality of taggants, are applied thereto. A system for applying taggants to goods such as cannabis is described in commonly assigned U.S. patent application Ser. No. 16/265,631, filed Feb. 1, 2019, and published as U.S. Pub. No. 2019/0241982, the entire contents of which are incorporated herein by reference. Such taggant application may be performed by a misting system, electrostatic spray system, atomized spray system, pressurized spray system, sprinkler system, fogging system, cooling system, handheld spray device, and powder dusting system. The taggant contains a unique nucleic acid signature, which can be used to identify the particular plant or group of plants. For example, cannabis plants grown at a particular authorized grower can have on or more taggant(s) applied thereto. The taggant contains one or more unique nucleic acid sequence(s). The unique sequence is stored in a database and associate with a particular grower. Therefore, when that unique nucleic acid sequence is detected, the plant can be verified as originating from that grower.

In practice, articles to be interrogated in accordance with system 10 are processed to obtain an article sample 16 thereof. This can be done by contacting the article with sample collection device 18 such as a standard swab as shown in FIG. 1. The sample obtained from the article 16 includes the nucleic acid taggent. The sample is then subjected to an on-site PCR amplification process wherein the nucleic acid in the taggant is replicated making it easier to detect. It is contemplated that the taggent may include one or more nucleic acid sequences. In order to begin the PCR amplification process, the collection device 18, is placed in a tube 20. Alternatively, a portion of the article, such as a thread, may be separated from the article and then placed directly in the tube 20. Liquid PCR amplification reagents 22 of the type know in the art for PCR amplification can then be added to the tube 20. All reagents can be packaged into droppers, which dispense proper quantities of materials. The contents in the tube are then added to the PCR amplification device 12 of a type known in the art, such as QP-1016-01, QP-1000-01 and QP-1000-01R marketed by miniPCR Bio™, or other suitable device. Such an amplification device 12 is relatively low cost, has a small footprint, and does not need to be plugged in to an external power source. The amplification device 12 can also operate at room temperature. The amplification device 12 may be connectable to a mobile communication device such as a smartphone or tablet via wireless communications protocols such as Bluetooth. This permits the PCR amplification to be performed in the field and at points of inspection. The PCR amplification can be limited to approximately 1 min per cycle or less depending on the thermocycling parameters. Once the PCR amplification is completed a PCR sample solution is obtained which carried therein the amplified sample of the nucleic acid sequences or sequences found in the sample.

Alternatively, a standard or an ultra fast PCR amplification device can be used such as one marketed by Molecular Biology Systems B.V. under the name NextGenPCR™ to reduce the processing time. While such an amplification device is not as portable as the device described above, it can be installed in an on-site laboratory.

After the PCR amplification process is performed, the amplified nucleic acid material may be processed by the Nucleic Acid Lateral Flow device (“NALF”) 14. In response to the detection of a particular target nucleic acid sequence, the NALF generates a visual result to help detect the nucleic acid sequence(s) that have been obtained from the article 16 and amplified. The NALF devices 14 can be made to detect a predetermined target nucleic acid sequence or sequences. For example, cannabis from a particular grower would be tagged with a taggent having a unique nucleic acid sequence. In order to verify the product as originating from a particular grower, a NALF would be prepared that can detect that particular sequence. In the field, if an inspector desires to confirm that the cannabis comes from a particular supplier, they would use the particular NALF which has been prepared to detect the nucleic acid sequence corresponding to that particular grower. Utilization of an imager device or a cell phone app can help store and track the data, along with a barcode to be integrated onto the NALF.

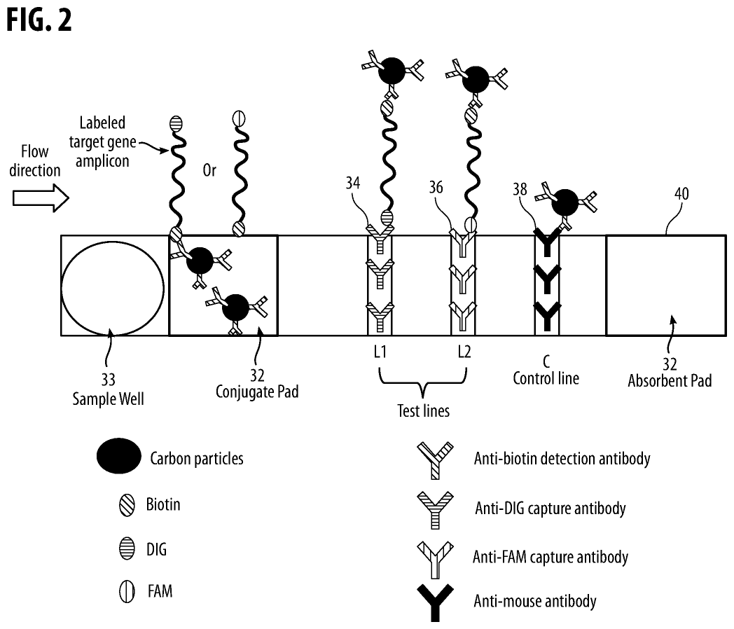

With reference to FIGS. 2 and 3A and B, a NALF device 14 includes a housing 30 that contains one or more elongate pads 32 to which a PCR output sample solution containing the nucleic acid is deposited in a sample well 33 located at a sample receiving end of the housing. The pads 32 are based on a series of capillary beds, such as pieces of porous paper, microstructured polymer, nitrocellulose membrane, or sintered polymer. Each of these pads 32 has the capacity to transport solution spontaneously. The sample pad 32 acts as a sponge and holds an excess of PCR sample solution. Once soaked, the PCR sample solution flows to an indication end having one or more test area(s) 34, 36 and the control area 38 which become visible through a window 39 in the housing 30. The test areas may produce a visual indicator, such as a line, upon detection of a predetermined nucleic acid sequence. The control area 38 may produce a visual indicator, such as a line, upon detection of the PCR sample solution indicating that the solution has traveled past the test area(s). While the term “test line” and “control line” are used in the disclosure, it is contemplated that the visual indicators could be of a variety of indicia or shapes such as lines, circles, dots, letters, numbers, etc. In one embodiment, the test areas may include test lines 34, 36 and the control area may include a test line 38 contain different biotin-modified nucleic acid probes grafted on streptavidin-functionalized nitrocellulose membrane support.

The nucleic acid sample is dropped on the sample pad 32 and mixed with conjugate probes. Target nucleic acids of the same sequence that have been hybridized with the conjugate probes will be further sandwich-hybridized with specific biotinmodified probes at the test line while the excess conjugate probes will be trapped with another specific biotin-modified probes at the control line.

The visual signal, i.e., activation of the test lines, originates from a sandwich hybridization assay occurring at the test line, at which a nucleic acid probe is commonly immobilized by a terminal biotin that interacts with streptavidin or NeutrAvidin on the nitrocellulose. As in lateral flow immunoassays, signaling moieties can be oligonucleotide-decorated gold or latex particles, or simply a fluorescent dye that modifies the tag sequence. For example, the visual results from a NALF testing a cannabis butane extract sample is shown in FIG. 3A.

When a test areas 34 and/or 36 is activated, it emits a signal, such as a visible colored stripe. The control line 38 is activated and emits a signal, such as a colored stripe, which indicates that the sample has flowed through and the material in the conjugate pad. After passing the test line and control line portion of the pads, the sample solution enters the final porous material that acts as a waste container 40.

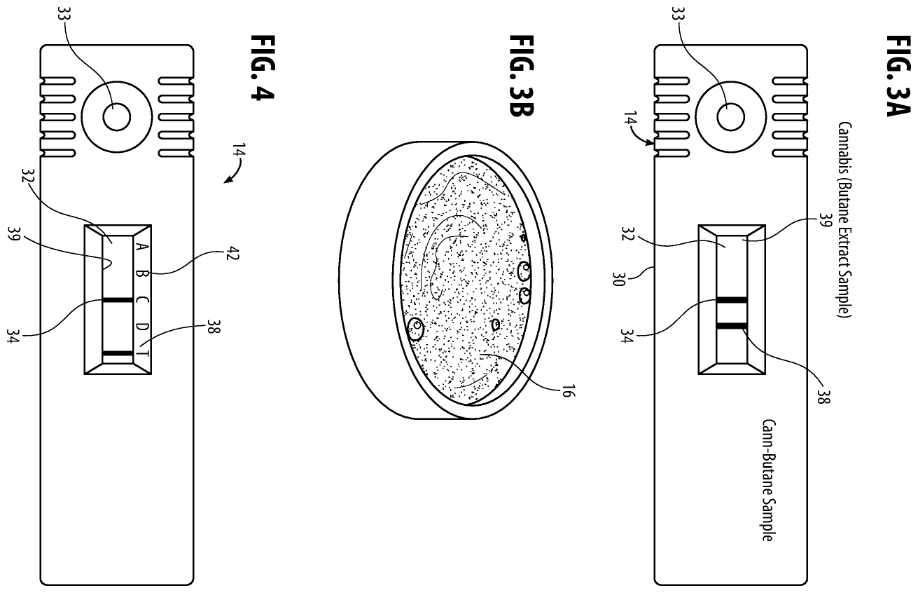

The test areas 34 and 36 are prepared to provide visual information when a particular nucleic acid sequence is detected. The sequence is associated with a particular aspect of the sample. For example, the test areas 34 and 36 may be activated and indicate whether a particular product came from a particular source. The test areas may also correlate to quality grade rating, and/or regulatory compliance data. In the case of cannabis products, the test area if activated could indicate that the product originated from a legal and licensed source. In addition, the test area of the pad may have a plurality of longitudinally spaced test lines sections with each being activated by a particular nucleic acid sequence. For example, with reference to FIG. 4, the plurality of test areas 34 could be used to indicate a particular geographical region, e.g., region A, B, C, or D. Each region would correspond to a particular nucleic acid sequence. The housing may have indicators 42 printed adjacent the window and where the various test lines would appear when activated. Therefore, for example, if test area C is activated and provides a colored line, for example, the user knows the sample was taken from a product originating in region C. Alternatively, the test areas could be formed so they each have a different color line or other indicia when activated, thereby providing an easy visual indication, e.g. blue is region A, red is region B, etc.

Accordingly, an aspect of the article 16 being inspected can be quickly and reliably verify using relatively inexpensive equipment. The training required to conduct the verification is minimal due to the nature of the process. In addition, there is no need to store the articles being inspected in a holding area while samples are sent to a remote lab and test results are pending.

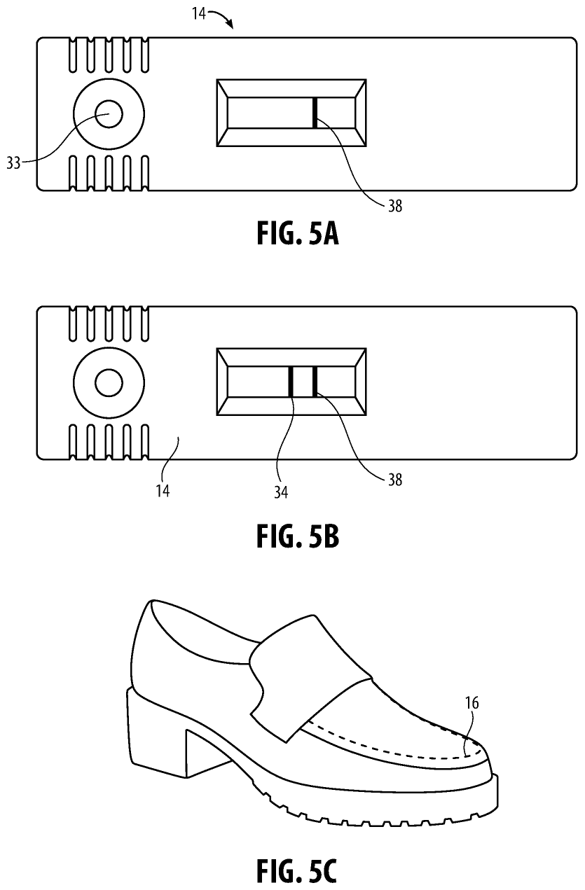

The use of the PCR device 12 along with a NALF 14 can be used on-site for a wide variety of authentication and verification applications. For example, with reference to FIGS. 5A-5C, commercial products such as clothing including a sewing thread 16 treated with a nucleic acid taggant. This permits manufactures to authenticate and validate their products anywhere within the supply chain by way of a common component, sewing thread. In the field, verification is possible using a portable PCR test and a NALF as described above.

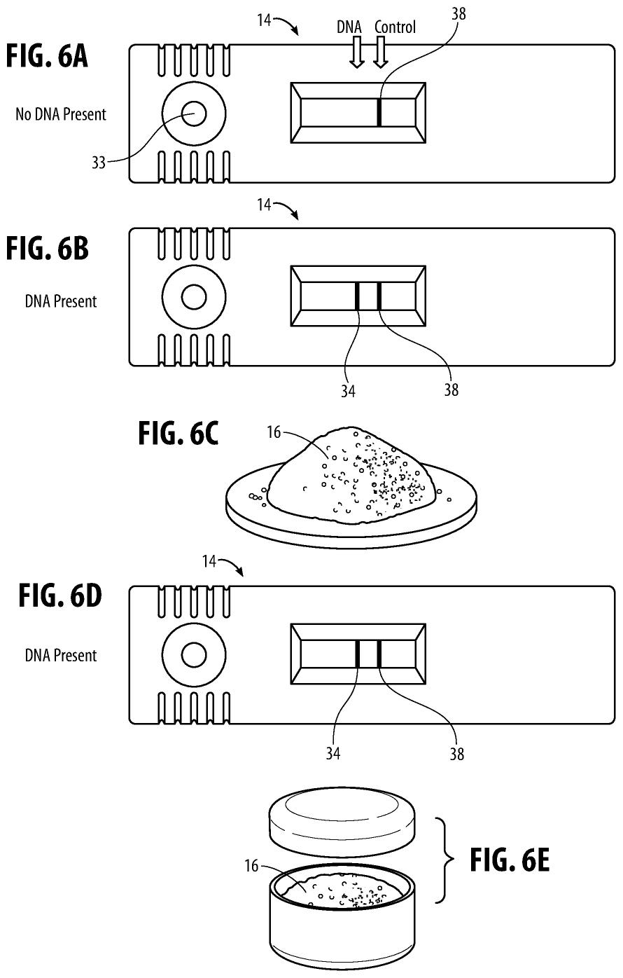

Likewise, cosmetics and/or cosmetic ingredients can be authenticated or verified in the field using the PCR test device 12 and a NALF device 14 as shown in FIGS. 6A-E. FIG. 6A shows a NALF wherein no predetermined target nucleic acid is detected from a sample. FIGS. 6B and 6C illustrate a NALF showing a detection of a target nucleic acid in a cosmetic ingredient and cosmetic product respectively.

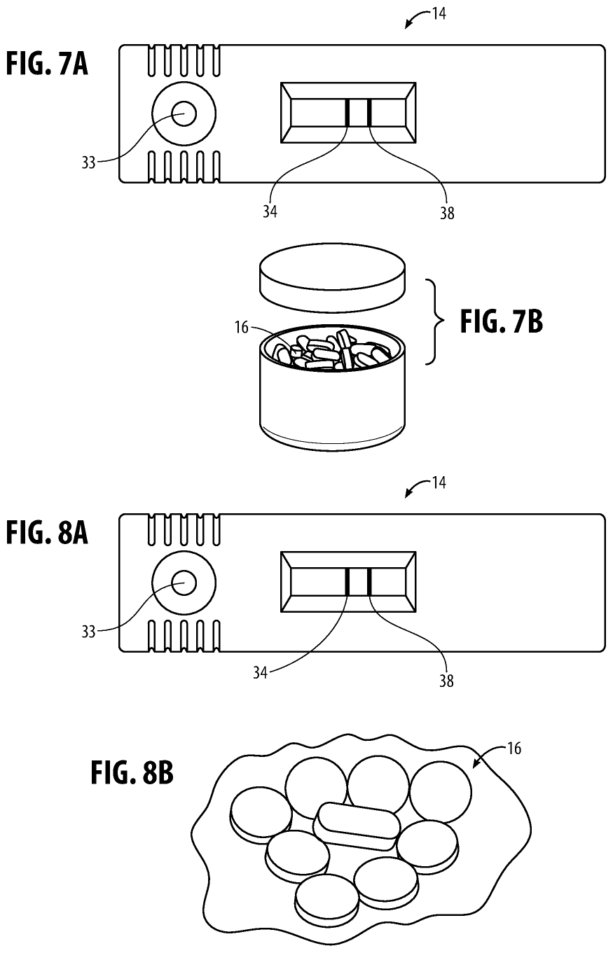

With reference to FIGS. 7A-B and 8A-B, nutraceuticals and pharmaceuticals, as well as ingredients for same, that have been treated with a taggant can also be authenticated and verified using system 10 with its PCR amplification device 12 and a NALF device 14 to provide a visual indication using a test line 34 and a control line 38.

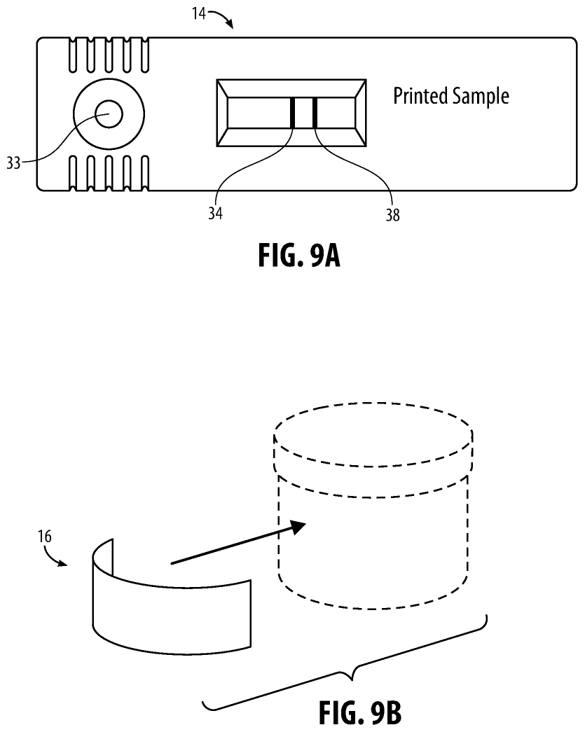

In addition, with reference to FIGS. 9A and 9B, a taggant can be added to commercially available labels attached to products or documents. These labels can be process to obtain a sample that is subjected to the PCR process and then the NALF device 14. The sample 16, therefore, can be interrogated using the system 10.

It is also contemplated that the system 10 and method described herein can be applied to other applications, for example, verifying cash and valuables in transit, print and packaging (Inkjet, thermal transfer, etc.), and fertilizer.

For example, system 10 permits for tracking the origin of cannabis and cannabis derivative products. A nucleic acid taggant having at least one predetermined nucleic acid sequence is applied to a cannabis plant to create a nucleic acid tagged cannabis plant. The taggant imparts at least one piece of information about the origin of the cannabis plant. For example it could be name or location of the grower. The nucleic acid tagged cannabis plant is then processed to create nucleic acid tagged cannabis products and/or nucleic acid tagged cannabis derivative products. When such products move through the stream of commerce they can be tracked based on the taggant. This is achieved by interrogating a nucleic acid tagged cannabis product and/or cannabis derivative product to ascertain information from the nucleic acid tag. The interrogation includes amplifying the nucleic acid contained on the sample by a polymerase chain reaction (PCR) device to produce an amplified PCR sample. The amplified PCR sample is deposited on a receiving end of a Nucleic Acid Lateral Flow (NALF) device to obtain a visual indication upon the detection of a first predetermined nucleic acid sequence.

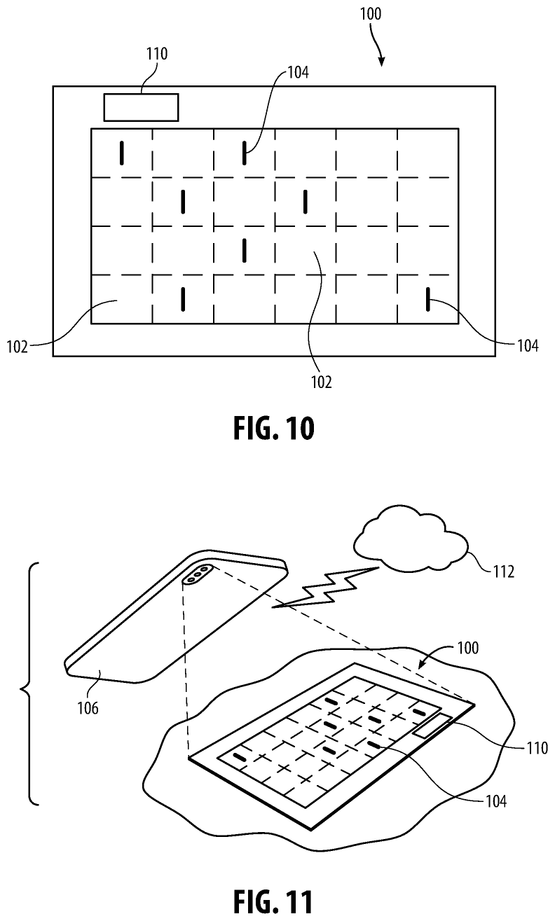

With reference to FIG. 10, it is further contemplated that a plurality of test areas 102 form a 2-dimensional NALF array 100. Each test area 102 is sensitive to a particular nucleic acid sequence. The predetermined nucleic acid sequence may correspond to a number of pieces of information about the article. For example, the sequence may be correspond to individual producer, quality grade rating, and/or regulatory compliance data. This correlation between the sequence and the information is stored in a database accessible by a user. This permits the user to obtain multiple pieces of data about the tagged article from a single sample taken for the article. One area in the array may provide geographical point of origin data, and other may provide the individual producer information, quality grade rating, and/or regulatory compliance data.

In an alternative embodiment, the article 16 may be marked by a plurality of taggants each having a unique nucleic acid sequence and/or a taggent including a plurality of nucleic acid sequences. Each unique sequence corresponds to particular piece of information about the article and that correlation is be stored on a database accessible by a user. For example, one taggent applied to the article may have a sequence from which the geographic location of the article can be obtained. Another taggent may have a sequence that indicates a particular produce of the product. The NALF array 100 would have test areas 102 that are sensitive to, and provide a visual signal 104 in response to, the particular nucleic acid sequences. When the PCR sample fluid is applied to the NALF array 100, the array will provide an visual indication when a particular nucleic acid sequence is sensed. Thus, multiple pieces of information about the tested article can be obtained by a user on site without having to send the sample to a lab.

For example, when a sample is applied to the NALF array 100, a number of different test areas 102 on the array will show a visual indication 104 based on the nucleic acid sequence or sequences found in the sample. This permits multiple pieces of information to be obtained from one sample. The various test areas 102 which are sensitive to the sample, will generate a visual indication 104, such as a color bar, or other indicia, once the array is treated with the sample. The array may have a number of visual bars 104 located at various positions in the array. A sample will trigger certain test areas 102 providing a pattern of indicator bars 104 as shown in FIG. 10. The user can then interpret the results and retrieve the data. In order to help facilitate the interpretation of the visual indicator(s), the array 100 with its pattern of visual indicators can then be compared to a chart which has the label proving information corresponding to each particular the indicator. The array 100 itself may have a unique code so the user may select the correct chart for the particular NALF array.

Alternatively, as shown in FIGS. 11-13, the interpretation of the visual results provided by the NALF array 100 can be provided by a computing device 106 running a software application. The user scans the treated array 100 using the computing device 106 such as a smartphone camera or other mobile computing device 106, and uploads the image to be processed by a software program running on the computing device 106. The NALF array 100 may include a visual tag 110 such as a bar code, QR code, or serial number The user may also scan this visual tag 110 so that the software knows what particular NALF array is being presented. The software may reside locally on the computing device 106 or in a remote network such as in the cloud 112 to which the computing device is wirelessly connected.

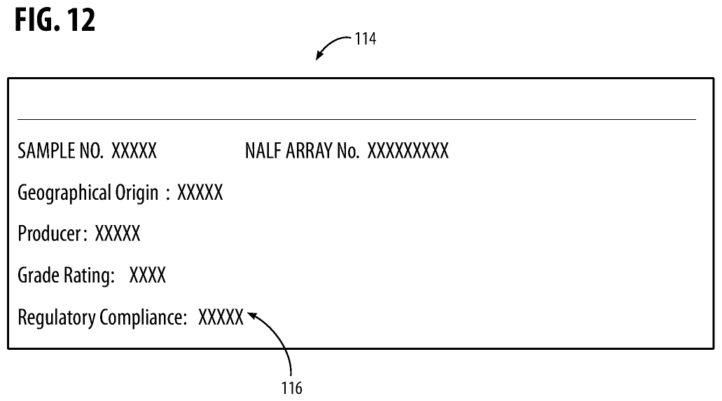

When the image of the treated array 100 is uploaded, the software compares the image of the particular NALF array with the images stored in a database. This database may be stored either on the device and/or on a remote server. Each stored image has stored with it data that provides information about the samples. The software will then match the scanned image with an image stored in the database. This can be achieved by using image recognition software of a type known in the art. The software then provides information about the sample on an output of the computing device 106. For example, the software will output on a display 114 to the user information 116 such as geographical origin, producer, quality grade rating, and regulatory compliance as shown in FIG. 12. A user at the point of inspection can then readily determine if the sampled article 16 meets the predetermined parameters.

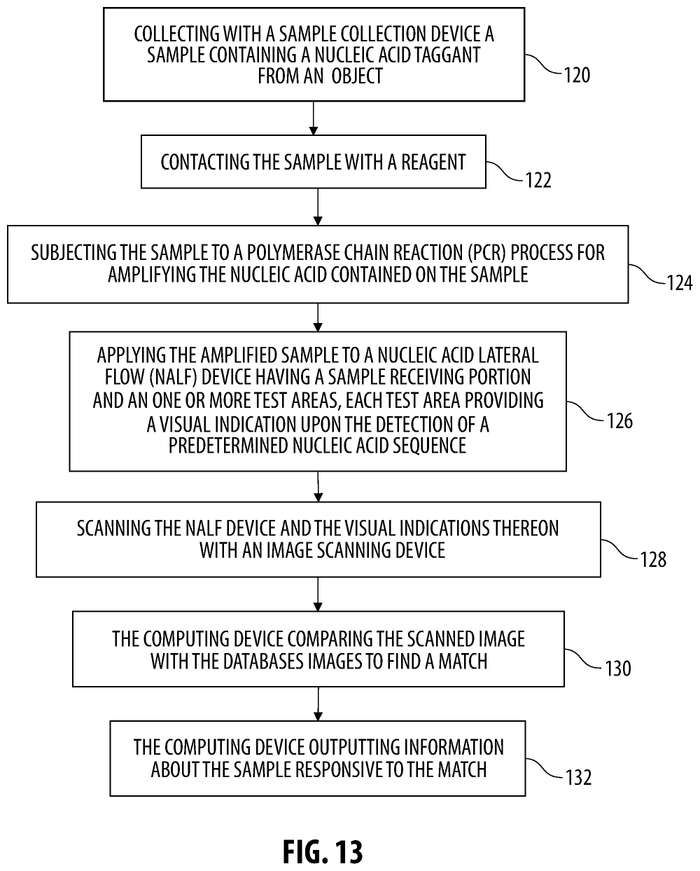

With particular reference to FIGS. 1 and 13, in operation, a user collects, with a sample collection device 18, a sample containing a nucleic acid taggant from an article or object 120. A sample is contacted with a reagent 122. The sample is subjected to a polymerase chain reaction (PCR) process for amplifying the nucleic acid contained on the sample to obtain an amplified sample 124. The amplified sample is applied to a Nucleic Acid Lateral Flow (NALF) device 14 having a sample receiving portion and one or more test area, each test area providing a visual indication upon the detection of a predetermined nucleic acid sequence 126. A user may then use this visual information to determine one or more properties of the article, such as its authenticity. In one embodiment, the NALF device and the visual indications thereon is scanned with an image scanning device 128. The scanning device being in operative communication with a computing device. The computing device is in communication with a database of images. The computing device compares the scanned image with the databases of images to find a match 130. The computing device outputs information to the user about the object responsive to the match. Such information about the article is stored in the database and correlates to the match. For example, if a predetermined nucleic acid sequence A is detected, information associated with sequence A can be obtained from a computer database stored in memory. Such information may include, for example, geographical origin, producer, quality grade rating, and/or regulatory compliance.

Although the disclosure provides specific examples, various modifications and changes can be made without departing from the scope of the disclosure as set forth in the claims below. Accordingly, the specification and figures are to be regarded in an illustrative rather than a restrictive sense, and all such modifications are intended to be included within the scope of the present disclosure. Any benefits, advantages, or solutions to problems that are described herein with regard to a specific example are not intended to be construed as a critical, required, or essential feature or element of any or all the claims.

What is claimed is:

1. A system for interrogating objects containing a nucleic acid taggant comprising:
a sample collection device for obtaining a sample containing at least one nucleic acid taggant from an object, the at least one taggent including at least one predetermined nucleic acid sequence;
a reagent for contacting the sample;
a polymerase chain reaction (PCR) device for receiving the sample and amplifying the at least one nucleic acid sequence contained in the sample to produce a PCR sample solution; and
a Nucleic Acid Lateral Flow (NALF) device having a sample receiving end for receiving the PCR sample solution, and having an indication end including a first test area and at least one control area, the first test area providing a visual indication upon the detection of the first predetermined nucleic acid sequence carried in the PCR sample solution.

2. The system as defined in claim 1, wherein the first test area visual indicator includes an indicia that becomes visible upon the detection of the first predetermined nucleic acid sequence.

3. The system as defined in claim 1, wherein the taggent includes a second predetermined nucleic acid sequence and the PCR device amplifies the second nucleic acid sequence, and the indication end includes a second test area which provides a visual indication upon the detection of the second predetermined nucleic acid sequence contained in the PCR sample solution, the second predetermined nucleic acid sequence being different for the first predetermined nucleic acid sequence.

4. The system as defined in claim 3, wherein the taggent includes a third predetermined nucleic acid sequence and the PCR device amplifies the third nucleic acid sequence, and the indication end includes a third test area which provides a visual indication upon the detection of a third predetermined nucleic acid sequence contained in the PCR sample solution, the third predetermined nucleic acid sequence being different for the first and second predetermined nucleic acid sequences.

5. The system as defined in claim 4, wherein the first, second, and third test areas each correlate with a geographical region.

6. The system as defined in claim 1, wherein the first test area correlates with a quality of the object.

7. The system as defined in claim 1, wherein the first test area correlates with regulatory compliance of the object.

8. The system as defined in claim 1, wherein the object is a plant-based product.

9. The system as defined in claim 8, wherein the plant-based product is cannabis.

10. The system as defined in claim 1, wherein the object is selected from a group consisting of a pharmaceutical and a nutraceutical.

11. The system as defined in claim 2, wherein the first and second test area provide visual indicators that differ in color from each other.

12. The system as defined in claim 1, wherein the sample is carried in the PCR sample solution and control area provides a visual indicia upon detection of the PCR sample solution.

13. A method of interrogating an object marked with a nucleic acid taggant comprising:
obtaining a sample from an object, the sample containing at least one nucleic acid taggant, the at least one taggent including at least one predetermined nucleic acid sequence;
placing the sample in a reagent;
amplifying the nucleic acid sequence contained in the sample by a polymerase chain reaction (PCR) device;
obtaining from the PCR device an amplified PCR sample solution; and
depositing the amplified PCR sample solution on a receiving end of a Nucleic Acid Lateral Flow (NALF) device, the NALF device having an indication end, the indication end including a first test area and at least one control area formed to detect a first predetermined nucleic acid sequence, the first test area providing a visual indication upon the detection of the first predetermined nucleic acid sequence in the PCR sample solution.

14. The method as defined in claim 13, wherein the object to be sampled is plant based.

15. The method as defined in claim 13, wherein the indication end includes a second test area which provides a visual indication upon the detection of a second predetermined nucleic acid sequence contained in the PCR sample solution, the second predetermined nucleic acid sequence being different for the first predetermined nucleic acid sequence.

16. The method as defined in claim 13, wherein the indication end includes a third test area which provides a visual indication upon the detection of a third predetermined nucleic acid sequence contained in the PCR sample solution, the third predetermined nucleic acid sequence being different for the first and second predetermined nucleic acid sequences.

17. The method as defined in claim 16, wherein the first, second, and third test areas each correlate with a geographical region.

18. The method as defined in claim 13, wherein the first test area correlates with a quality of the object.

19. The method as defined in claim 18, wherein the first test area correlates with regulatory compliance of the object.

20. A method of tracking the origin of cannabis and cannabis derivative products comprising:
applying at least one nucleic acid taggant having at least one predetermined nucleic acid sequence to a cannabis plant to create a nucleic acid tagged cannabis plant, the at least one taggant imparting at least one piece of information about the origin of the cannabis plant;
processing the nucleic acid tagged cannabis plant to create nucleic acid tagged cannabis products and/or nucleic acid tagged cannabis derivative products;
interrogating a nucleic acid tagged cannabis product and/or cannabis derivative product to ascertain information from the nucleic acid tag; the interrogation including:
amplifying the nucleic acid contained on the sample by a polymerase chain reaction (PCR) device to produce an amplified PCR sample including a first predetermined nucleic acid sequence; and
depositing the amplified PCR sample on a receiving end of a Nucleic Acid Lateral Flow (NALF) device to obtain a visual indication upon the detection of a first predetermined nucleic acid sequence.

21. A system for interrogating objects containing a nucleic acid taggant comprising:
a sample collection device for obtaining a sample containing at least one nucleic acid taggant from an object, the nucleic acid taggent including at least one predetermined nucleic acid sequence;
a reagent for contacting the sample;
a polymerase chain reaction (PCR) device for receiving the sample and amplifying the nucleic acid contained on the sample to produce a PCR sample solution;
a Nucleic Acid Lateral Flow (NALF) device having a sample receiving portion for receiving the PCR sample solution and one or more test areas, each test area providing a visual indication upon the detection of a predetermined nucleic acid sequence carried in the PCR sample solution;
a computing device being in communication with an image scanning device for scanning the NALF device and the visual indications thereon; and
the computing device being in communication with a database of images, the computing device comparing the scanned image with the databases images to find a match, the computing device outputs information about the object from which the sample is obtained responsive to the match.

22. A method for interrogating objects containing a nucleic acid taggant comprising:
collecting with a sample collection device a sample containing at least one nucleic acid taggant from an object, the at least one taggent including at least one predetermined nucleic acid sequence;
contacting the sample with a reagent;
subjecting the sample to a polymerase chain reaction (PCR) process for amplifying the at least one predetermined nucleic acid sequence contained in the sample to obtain an amplified PCR sample;
applying the amplified PCR sample to a Nucleic Acid Lateral Flow (NALF) device having a sample receiving portion and an one or more test areas, each test area providing a visual indication upon the detection of the predetermined nucleic acid sequence;
scanning the NALF device and the visual indications thereon with an image scanning device, the scanning device being in operative communication with a computing device;
the computing device being in communication with a database of images, the computing device comparing the scanned image with the databases images to find a match; and
outputting information with the computing device about the object responsive to the match.

23. The method as defined in claim 22, wherein the sample includes a second predetermined nucleic acid sequence;
